(19) United States
(12) Patent Application Publication
(10) Pub. No.: US 2026/0108162 A1
(43) Pub. Date: Apr. 23, 2026

(54) NON-INVASIVE CENTRAL VENOUS PRESSURE MONITORING DEVICE

(71) Applicant: HADASIT MEDICAL RESEARCH SERVICES AND DEVELOPMENT LTD., Jerusalem (IL)

(72) Inventors: Offer AMIR, Jerusalem (IL); Haytam KASEM, Jerusalem (IL); Kareem ABD-RBO, Jerusalem (IL); Aiman ABU AMMAR, Jerusalem (IL); Dean NACHMAN, Jerusalem (IL)

(21) Appl. No.: 19/424,801

(22) Filed: Dec. 18, 2025

Related U.S. Application Data
(63) Continuation of application No. PCT/IL2024/050605, filed on Jun. 20, 2024.
(60) Provisional application No. 63/522,437, filed on Jun. 22, 2023.

(51) Int. Cl. A61B 5/0215; A61B 5/00; A61B 5/021; A61B 90/00

(52) U.S. Cl. CPC A61B 5/02152; A61B 5/02141; A61B 5/442; A61B 5/6833; A61B 5/685; A61B 5/7271; A61B 90/06; A61B 2090/064; A61B 2562/0247; A61B 2562/0261; A61B 2562/028; A61B 2562/164

(57) ABSTRACT

This invention relates to a method and system for non-invasively monitoring central venous pressure, using a set of microneedles and micro-electromechanical systems, e.g., in heart failure. Herein, the microneedle-based sensor attaches to the skin at the neck of a subject to monitor central venous pressure, as it is a direct representative of filling pressures causing skin sub-surface strain.

### CROSS REFERENCE TO RELATED APPLICATIONS

This application is a Bypass Continuation of PCT Patent Application No. PCT/IL2024/050605 having International filing date of Jun. 20, 2024, which claims the benefit of priority of U.S. Provisional Ser. No. 63/522,437, filed Jun. 22, 2023, the contents of which are all incorporated herein by reference in their entirety.

### FIELD OF THE INVENTION

This invention relates to a method and system for non-invasively monitoring central venous pressure.

### BACKGROUND OF THE INVENTION

Heart failure (HF) is a common clinical syndrome with detrimental effects on individual patients and society levels. 2.2% of US adults or nearly 6.2 million individuals suffer from HF, employing a significant yearly financial burden estimated at 30.7 billion US Dollars in 2012, projected to more than double by 2030. The prevalence of heart failure is expected to continuously grow due to medical and societal developments. Recently, advances in heart failure drug and device-based treatments brought impactful achievements to the field, yet a quarter of patients will endure considerable symptoms, hospitalizations, and mortality despite optimal medical treatment. Consequently, a different approach to the management of heart failure is essential.

Heart failure is characterized by a progressive course of disease accompanied with recurrent exacerbations leading to high hospitalization and rehospitalization rates, accounting for a substantial part of the disease load. Due to the high prevalence and financial incentive, reducing Acute Decompensated Heart failure (ADH) hospitalization and readmission rates has become a foremost priority in health systems. The pathophysiological process of ADH begins days or even weeks prior to the development of noticeable signs and symptoms. Understanding the time course for the progress to ADH, it has been postulated that interventions prior to overt clinical manifestation to achieve euvolemic status may prevent exacerbation.

Elevated filling pressure in the right and left sides of the heart is the key for acute heart failure decompensation. Accordingly, monitoring and tracking in real-time manner an increase in the cardio-vascular system pressures may warn of pending decompensation and heart failure admission as well as guide therapy and improve both quality and life span of the heart failure patients.

One site, which is a traditional site to evaluate right (reflecting also to the left) intra-cardiac pressure, is the central venous pressure. Right atrial pressure measurement (RAP), also referred to as central venous pressure (CVP) is commonly assessed in an invasive procedure with significant potential complications such as vascular, cardiac or pulmonary injuries. It also requires special procedure, tools, and intensive care settings. Obviously, ambulatory and home measurements are not practical. The neck veins (jugular veins) are an acceptable surrogate for CVP measurement, when the veins are distended an elevated pressure is presumed. Nevertheless, there is no objective, let alone ambulatory, manner for the measurement of this pressure.

Therefore, there is a need for a method and system for non-invasively monitoring central venous pressure.

### SUMMARY OF THE INVENTION

This invention relates to a method and system for non-invasively monitoring central venous pressure.

Some embodiments relate to a device for essentially non-invasively monitoring a subject's blood vessel pressure (for example, central blood vessel pressure, more specifically, central venous pressure), which may include a microneedle structure comprising a first surface, a second surface opposing said first surface, and a plurality of microneedles attached to or being an integral part of the first surface, wherein the plurality of microneedles is configured to penetrate a skin surface at a target area above and/or in vicinity to the subject's blood vessel pressure (for example, central blood vessel pressure, more specifically, central venous pressure); and at least one force sensor associated with or incorporated within the microneedle structure, wherein the at least one sensor is configured to provide signals indicative of the central venous pressure.

There is provided herein, in accordance with some embodiments, a system for essentially non-invasively monitoring a subject's central venous pressure, the system includes:
a microneedle structure including a first surface and a second surface opposing said first surface, wherein the microneedle structure further includes a plurality of microneedles attached to or integrated within the first surface thereof, and wherein the plurality of microneedles is configured to penetrate a skin surface at an area above and/or in vicinity to the subject's central vein; at least one sensor associated with or incorporated within the microneedles and/or the microneedle structure, wherein the at least one sensor is configured to provide pressure indicative signals; and
a patient data acquisition processing system configured to receive data from the at least one sensor and to apply trained algorithms (for example, AI (e.g., machine learning) based algorithms) on the received pressure signals, and to provide values indicative of the central venous pressure of the subject.

According to some embodiments, the patient data acquisition processing system is directly or remotely (e.g., cloud based) connected to the at least one sensor.

As used herein, in accordance with some embodiments, the term “essentially non-invasively” may refer to a procedure that does not involve penetration of a device (for example, needles) deeper than the skin.

According to some embodiments, the device may include a flexible pad configured to conform the microneedle structure to contours of the subject's target area. According to some embodiments, the flexible pad may be adhered to and/or positioned in proximity to the second surface of the microneedle structure.

According to some embodiments the device may include an adhesive layer. According to some embodiments, the adhesive layer may be a double adhesive layer located between the second surface of the microneedle structure and a first surface of the flexible pad. According to some embodiments, the adhesive layer may extend over and beyond a second surface of the flexible pad and may be configured to attach the device to the subject's skin.

According to some embodiments, the device may include a rigid substrate positioned at the base of the device. According to some embodiments, the rigid substrate may be configured to generate contralateral pressure to the venous pressure and increase the sensor sensitivity.

According to some embodiments, the device may be associated with a patient data acquisition processing system configured to receive data from at least one sensor. According to some embodiments, the patient data acquisition processing system may be directly or remotely connected to at least one sensor. According to some embodiments, the patient data acquisition system may include a processor.

According to some embodiments, the at least one force sensor may be a force sensor, a pressure sensor or a strain gauge. Optionally, the sensor may be a force resistance sensor. According to some embodiments, the at least one sensor may be configured to detect skin sub-surface strain, skin fluctuations that result from vascular changes in diameter due to variation of internal pressure, skin stretching, or a combination thereof.

According to some embodiments, the at least one force sensor may be polymer-based sensors, specifically electroactive and flexible polymers. According to some embodiments, such polymers be customized for enhanced properties as required. Examples of such polymers may include, Poly(3,4-ethylenedioxythiophene) (PEDOT), Polypyrrole (PPy), Polyaniline (PANI), Polythiophene (PTh), Poly(3-hexylthiophene) (P3HT) or any combination thereof. Each possibility is a separate embodiment.

According to some embodiments, the polymers may include co-polymers, for example, poly(3,4-ethylenedioxythiophene):tosylate (PEDOT:TOS), PEDOT-co-polyurethane (PEDOT-co-PU), PEDOT-co-polylactic acid (PEDOT-co-PLA)′ Polyaniline-co-polypyrrole (PANI-co-PPy), PEDOT-co-polycaprolactone (PEDOT-co-PCL), PEDOT-co-polyethylene glycol (PEDOT-co-PEG) or any combination thereof. Each possibility is a separate embodiment.

According to some embodiments, the device may include a casing configured to house the microneedle structure and at least one sensor. According to some embodiments, the casing may include a flexible pad, a rigid substrate, a connection to a patient data acquisition processing system or any combination thereof.

According to some embodiments, the density of the microneedles on the first surface of the microneedle structure is between about 5 to about 150 microneedles/cm² (for example, about 5-20, about 10-50, about 20-80). According to some embodiments, at least one component of the microneedles, microneedle structure and at least one sensor is 3D printed. According to some embodiments, at least one force sensor may be integrated within the microneedles to provide a combined microneedle-mechanical sensor.

According to some embodiments, the central blood vessel may include a central vein, for example, the (left and/or right jugular vein), a femoral vein, a superior vena cava, an inferior vena cava, or other monitoring site. According to some embodiments, the device may be for use in monitoring a patient's volume status, and/or pathologies characterized by changes in central venous pressure. According to some embodiments, the pathologies characterized by changes in central venous pressure is selected from a group consisting of: right heart failure, heart failure, pulmonary embolism, pulmonary hypertension, cardiac tamponade, pericardial tamponade, hemodynamic shock, and valvular disorders. Each possibility is a separate embodiment.

According to some embodiments, the at least one force sensor may be a pressure sensor, a strain gauge sensor, a piezoelectric sensor, tactile sensor or any other appropriate sensor. Each possibility is a separate embodiment.

According to some embodiments, there is provided herein a method for essentially non-invasively monitoring the central venous pressure of a subject, the method includes: placing a central venous pressure monitoring device to the subject's skin surface at an area above and/or in vicinity to the subject's central vein, wherein the device includes: a microneedle structure comprising a first surface, a second surface opposing said first surface, and a plurality of microneedles attached to or being an integral part of the first surface thereof; and at least one force sensor in association with or incorporated within the microneedles and/or the microneedle structure; piercing the skin surface with the microneedles; measuring skin deformation with the at least one sensor; obtaining, using a data acquisition processing system, skin deformation related signals; applying, using the data acquisition processing system, trained algorithms on the received signals; and providing values indicative of the central venous pressure of the subject, based on the obtained signals and the trained algorithms.

Without limiting to any theory, in accordance with some embodiments, the skin deformation may be due to venous pressure variation as arise from changes in the diameter of the vessel and the strain of the adjacent skin.

According to some embodiments, the signals may be transmitted to a patient data acquisition processing system. According to some embodiments, the transmitting to the patient data acquisition processing system may be by a direct or indirect connection to the at least one sensor.

According to some embodiments, the method may provide a pressure profile of the subject. According to some embodiments, the pressure profile may be provided to a medical professional for real-time monitoring and/or periodic review.

According to some embodiments, the system may alert a user, a carer, or a medical professional when the central venous pressure monitoring device detects a pre-defined change in one or more measured and/or calculated parameters. According to some embodiments, the change in one or more measured and/or calculated parameters is above and/or below a pre-defined threshold for the parameter. According to some embodiments, the device may be placed (e.g., adhered) by self-application, or application by a carer, or medical professional.

According to some embodiments, the monitoring may be configured for one time monitoring, short term monitoring, or long-term monitoring. According to some embodiments, the monitoring may be monitoring any one of fluctuations in venous pressure, and specific pressure waveforms arising from the central blood vessel, reflecting intra-cardiac pressures. According to some embodiments, the non-invasively monitoring may detect an arterial pressure wave, monitors blood pressure, and/or integrates arterial and venous data.

According to some embodiments, the method may further include predicting central venous pressure based on measured sub-surface skin deformation and a trained algorithm model (e.g., AI based, for example, machine learning based algorithm model).

According to some embodiments, the method may further include predicting central venous pressure based on measured sub-surface skin deformation and time series regression algorithm model.

It is noted that, although reference may be made specifically to monitoring central venous pressure, the scope of this disclosure covers device, system of methods for monitoring any type of blood vessel, for example, arteries, veins, central arteries and central veins.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some, or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Examples illustrative of embodiments are described below with reference to figures attached hereto. In the figures, identical structures, elements, or parts that appear in more than one figure are generally labeled with the same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIGS. 1A and B schematically illustrate a preferred target area for pressure sensing using a central venous pressure monitoring device, in accordance with some embodiments;

FIG. 2 schematically illustrates a side view of a central venous pressure monitoring device, in accordance with some embodiments;

FIGS. 3A and 3B schematically illustrate a side view, perspective view and cut away view, respectively, of a central venous pressure monitoring device, in accordance with some embodiments;

FIG. 4 is a schematic illustration of a central venous pressure monitoring device, in accordance with some embodiments;

FIGS. 5A and 5B are schematic illustrations of cross section of a central venous pressure monitoring device, in accordance with some embodiments;

FIG. 5C is a schematic illustration of an isometric partial view of a central venous pressure monitoring device, in accordance with some embodiments;

FIG. 6 is a schematic illustration of an isometric partial view of a central venous pressure monitoring device, in accordance with some embodiments;

FIG. 7 is an exemplary block diagram of a central venous pressure monitoring device, in accordance with some embodiments;

FIG. 8 is an exemplary block diagram of a central venous pressure monitoring system, in accordance with some embodiments;

FIGS. 9A and 9B are exemplary photographs of an artificial neck vein model, in accordance with some embodiments;

FIG. 10 schematically illustrates an upper view of a vein model with the different test locations of the monitor, in accordance with some embodiments;

FIG. 11 is a pressure profile of a central test location of the model flow system using a central venous pressure monitoring device, in accordance with some embodiments;

FIG. 12 is a pressure profile of a back test location of the model flow system using a central venous pressure monitoring device, in accordance with some embodiments;

FIG. 13 is a pressure profile of a right test location of the model flow system using a central venous pressure monitoring device, in accordance with some embodiments;

FIG. 14 is a pressure profile of a front-left test location of the model flow system using a central venous pressure monitoring device, in accordance with some embodiments;

FIG. 15A illustrates the development of a time series regression model for predicting central venous pressure, in accordance with some embodiments;

FIG. 15B illustrates a graph of true venous pressure readings compared to the algorithm's prediction as derived from the sensor reading using a simulator that integrates a model of the carotid arterial and jugular venous vessels; and

FIG. 16 is an illustrative flowchart of a method for monitoring central venous pressure monitoring device, in accordance with some embodiments.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Some embodiments relate to a method and system for non-invasively monitoring central venous pressure. According to some embodiments, the system may include a non-invasive cardiac filling pressure measurement device. According to some embodiments, the device may be used for monitoring pathologies characterized by changes in central venous pressure (CVP) include heart failure, pulmonary embolism, pericardial tamponade, hemodynamic shock, right heart failure and valvular disorders or diseases. Additionally, the management of these pathologies may be facilitated by central venous pressure monitoring.

According to some embodiments, the device for monitoring central venous pressure may include one or more microneedles and a micro-electromechanical system including one or more sensors, e.g., force sensors. According to some embodiments, the device may facilitate non-invasive direct monitoring of central venous pressure, e.g., at the jugular vein. Advantageously, the device may facilitate more sensitive and less cumbersome monitoring of central venous pressure. Additionally, advantageously the microneedles may be essentially minimally invasive, since microneedles create micron sized pores in the skin avoiding deeper layers of the dermis where higher concentrations of nerves exist. Therefore, microneedles may be used for transdermal sensing as long-term wearable health monitoring devices. Further advantageously, the device may be convenient to apply, e.g., for self-application, application by minimally trained carers (e.g., family members, etc.), application by medical personnel, etc. Additionally, and/or alternatively, such a medical device may include additional functionality to enable seamless data transfer from the patient to the physician, e.g., leveraging modern technology, such as smartphones, etc.

According to some embodiments, the device may be small, with dimensions ranging between about 0.5 cm to about 7 cm in diameter and a height of about 0.3 cm to about 2 cm of active sensor surface accompanied with surrounding medical grade bandage, adhesive pad, surgical tape, etc.

According to some embodiments, the microneedles may have a height ranging between about 50 μm to about 2,500 μm, between about 100 μm to about 2,000 μm, between about 150 μm to about 1,500 μm, or between about 200 μm to about 1,000 μm. Each possibility is a separate embodiment.

According to some embodiments, the microneedles may have a base width ranging between about 15 μm to about 750 μm, between about 25 μm to about 500 μm, between about 50 μm to about 250 μm, or between about 75 μm to about 200 μm. Each possibility is a separate embodiment.

According to some embodiments, the microneedles may have a base diameter ranging between about 50 μm to about 300 μm, between about 50 μm to about 100 μm, between about 50 μm to about 200 μm, or between about 100 μm to about 300 μm. Each possibility is a separate embodiment.

According to some embodiments, the density of microneedles on a microneedle structure may be ranging between about 1 to about 10 microneedles/cm², between about 10 to about 50 microneedles/cm², between about 50 to about 100 microneedles/cm², between about 50 to about 150 microneedles/cm², between about 100 to about 250 microneedles/cm², or between about 250 to 500 microneedles/cm². Each possibility is a separate embodiment.

According to some embodiments, the microneedles may be manufactured using a number of different methods, e.g., 3D printing technologies, photolithography, droplet-born air blowing (DAB) method, micro-molding, solvent casting, one or two step casting into negative templates, thermal melting methods, etc. and/or a combination thereof. Each possibility is a separate embodiment.

According to some embodiments, the microneedles may be hollow. According to some embodiments, the microneedles may be composed at least in part of hollow metallic needles. According to some embodiments, the microneedles may be manufactured by common techniques in the fabrication, such as photochemical etching, electroplating, and laser cutting, micro-computer numerical control (CNC) cutting, etc.

According to some embodiments, the microneedles may be composed, at least in part, of one or more bio-compatible polymers. According to some embodiments, the bio-compatible polymers may be conductive. According to some embodiments, the conductive polymers may have electro-mechanical sensing properties. According to some embodiments, the bio-compatable polymers may be a synthetic polymer e.g., polylactic acid (PLA), poly(lactic-co-glycolic acid) (PLGA), polycaprolactone (PCL), polystyrene etc., or a natural polymer e.g. cross-linked chitosan, alginate, hyaluronic acid, etc., and/or any combination thereof. Each possibility is a separate embodiment.

According to some embodiments, the microneedles may be composed of one or more polymers which may change their resistance as a result of mechanical strains, and/or modified by sectioning the microneedle bases. According to some embodiments, in case the microneedles are composed of the polymer, the polymer is a bio-compatible polymer.

According to some embodiments, the microneedles and/or their supporting structure may advantageously be conformable for use on curved skin and/or may react based on the skin curvatures in a single axis, in plane, or 3D manner.

According to some embodiments, the microneedles may themselves be used as high-resolution 2D or 3D mechanical sensors. According to some embodiments, the microneedles may be incorporated into a flexible matrix material to provide a flexible matrix of microneedles. According to some embodiments, the flexible microneedles may be integrated with one or more strain sensing elements, which may change their resistance or capacitance when bending, stretching, compressing forces and/or a combination thereof are applied.

According to some embodiments, the microneedles may be configured to provide one or more therapeutic agents. According to some embodiments, the microneedles may be configured for injection of one or more therapeutic agents. According to some embodiments, the microneedles may be configured for controlled release of one or more therapeutic agents. According to some embodiments, the microneedles may enable a full close loop system, wherein one or more therapeutic agents may be released following a detected signal.

According to some embodiments, one or more of the mechanical and/or supporting components of the monitoring device may be designed and/or printed using a 3D printer, cast into negative template, prepared using microlithography, laser machining processes, extrusion, injection molding, etc. and/or a combination thereof. Each possibility is a separate embodiment.

According to some embodiments, the microneedles may be attached to a first surface of a microneedle structure. According to some embodiments, the microneedle structure may include and/or be associated with one or more additional components. According to some embodiments, the components may be arranged in any order.

According to some embodiments, a second surface of a microneedle structure may include and/or may be associated with an adhesive layer. Optionally, the adhesive layer may include an adhesive, e.g., a polyurethane adhesive, acrylics, silicone, etc. Optionally, the adhesive may be hypoallergenic. Optionally, the adhesive layer may be a dual adhesive layer. Optionally, a first surface of the adhesive layer may be associated with the skin of a subject.

According to some embodiments, the adhesive layer may be an outermost layer (layer distal to the skin) associated with the device, e.g., a patch, which may be placed over the device. According to some embodiments, the adhesive layer may extend over and beyond a second surface of the flexible pad. According to some embodiments, the adhesive layer may be configured to attach the device to a subject's skin.

According to some embodiments, the device may include both an adhesive associated with a second surface of a microneedle structure and an outer adhesive layer. According to some embodiments, an adhesive layer may be configured to prevent and/or reduce infection. According to some embodiments, an adhesive layer may be configured to improve and/or sustain adhesion of the device to the user's skin. According to some embodiments, an adhesive layer may be configured to reduce and/or prevent movement of the device after application to a subject's skin. Optionally, a flexible pad may be associated with a second surface of an adhesive layer, e.g., extending over and beyond a second surface of the flexible pad and is configured to attach the device to the subject's skin.

According to some embodiments, the adhesive layer may be a patch. Optionally, the patch and/or part thereof may be water-resistant and/or waterproof. Optionally, the patch and/or part thereof may be hypoallergenic. Optionally, the patch and/or part thereof may include a polyurethane adhesive, acrylics, silicone, etc. Optionally, the patch may be configured to provide a measurable counter response to the skin stretching and/or strain.

According to some embodiments, the second surface of the microneedle structure may be associated with a flexible pad, e.g., silicone, etc. According to some embodiments, the flexible pad may comprise a plasticizer, e.g., polyols (such as, glycerol, polyethylene glycols) or organic esters (such as, diethyl phthalate (DEP) and triethyl citrate (TEC)), etc. According to some embodiments, the flexible pad may be configured to conform to contours of the user's skin. According to some embodiments, a first surface of the flexible pad may be associated with an adhesive layer and/or a microneedle support. According to some embodiments, a second surface of the flexible pad may be associated with a rigid substrate and/or one or more sensors, e.g., force sensors.

According to some embodiments, the device may include a rigid substrate. Optionally, the rigid substrate may be 3D printed or cast into a negative template. According to some embodiments, the rigid substrate may serve as a counter support for the force sensor on the side distal to the force sensor, such as, the base of the device.

According to some embodiments, the rigid substrate may be configured to generate contralateral pressure to the venous pressure. According to some embodiments, the rigid substrate may be configured to increase the sensor sensitivity.

According to some embodiments, the device may include a casing. Optionally, the casing may be 3D printed. Optionally, the casing may be water-resistant and/or waterproof. Optionally, the casing may include one or more mechanical, and/or electronic components, e.g., a processor, a wi-fi connector, Bluetooth connector, cable, sensors, printed circuit boards (PCBs), flexible PCB, optic parts, etc. and/or any combination thereof. Each possibility is a separate embodiment.

According to some embodiments, the device may be associated with and/or may include one or more sensors. According to some embodiments, the sensor may be a pressure sensor, a force sensor, a strain gauge, a flex sensor, a bend sensor, an optic sensor, etc. and/or a combination thereof. Each possibility is a separate embodiment. According to some embodiments, the sensor may be a Force Sensing Resistor (FSR). According to some embodiments, the sensor may be configured to be sensitive enough to measure changes in venous pressure. According to some embodiments, the sensor may be configured to measure small variations in skin sub-surface strain.

According to some embodiments, the sensors may be integrated with the plurality of microneedles to provide a combined microneedle-mechanical sensor.

According to some embodiments, the monitoring device may be attached to a subject's skin at the neck. According to some embodiments, the monitoring device may be configured to measure central venous pressure of the jugular vein, as it is a direct representative of filling pressures. Optionally, the monitoring device may measure skin sub-surface strain. According to some embodiments, the microneedles may pierce through the stratum corneum to better detect sub-surface skin deformation due to jugular vein pressure variation.

According to some embodiments, the profile of pressure variation may be processed by, and/or emitted to a patient data acquisition processing system. According to some embodiments, the monitoring device may be connected directly (e.g., by a cable, etc.) and/or indirectly (e.g., wirelessly by wi-fi, Bluetooth, etc.) to a patient data acquisition processing system. According to some embodiments, the patient data acquisition processing system may be a processor within the monitoring device. According to some embodiments, the patient data acquisition system may include a remote processor.

According to some embodiments, the patient data acquisition system processing may provide the acquired data visually and/or electronically to a visualization device, e.g., a screen, printer, etc. Optionally, the patient data acquisition processing system may provide the acquired data to an on-site visualization device. Optionally, the patient data acquisition processing system may provide the acquired data to an off-site medical professional for real-time monitoring and/or periodic review (e.g., hourly, twice daily, daily, weekly, bi-weekly, monthly, etc.). Optionally, the patient data acquisition processing system may save the acquired data, e.g., in a memory storage device, cloud, etc. Optionally, the memory storage device may be incorporated within the casing of the device. Optionally, the memory storage device may be remote to the device.

According to some embodiments, the patient data acquisition processing system may provide an alert to the user, a carer and/or a medical professional when the monitoring device detects a pre-defined change in one or more measured and/or calculated parameters, e.g., central venous pressure (CVP) profile, blood pressure changes, variations in the CVP wave morphology, etc. For example, elevation in CVP may indicate volume over-load in patients with heart failure and necessitate the up titration of diuretic medications. In the opposite case, decreased CVP pressure may indicate hypovolemia and may necessitate a reduction in diuretic prescription. Additionally, CVP monitoring may facilitate diagnosis of the above-mentioned medical conditions, follow-up on patients with the above-mentioned medical conditions, aid in risk stratification, and/or treatment adjustments. Optionally, the patient data acquisition processing system may provide an alert to the user, a carer and/or a medical professional when one or more monitored parameters is measured and/or calculated to be above and/or below a pre-defined threshold. Optionally, a medical professional may pre-define one or more parameters and/or thresholds based on a user's medical history and/or condition.

According to some embodiments, the monitoring device may be self-applicable, applicable by a carer and/or medical professional. According to some embodiments, the monitoring device may be applied for immediate monitoring, short term monitoring (e.g., several hours, days, etc.) and/or long-term monitoring (e.g., several days, a week, several weeks, etc.).

Advantageously, the monitoring device may be simple to apply and/or remove, without the need for a special procedure, tools and/or an intensive care setting. According to some embodiments, the monitoring device may allow for clinical, outpatient and/or home-care application. According to some embodiments, the monitoring device may allow for ambulatory use.

According to some embodiments, the monitoring device may be attached to the skin at the area of the neck veins, preferably targeting the internal jugular vein. According to some embodiments, the monitoring device may monitor the fluctuations which may arise from the amplitude of the pressure wave and/or the height in the skin of the neck where the pressure wave is noticeable, and/or the specific pressure waveforms arising from the jugular vein, reflecting intra-cardiac pressures. Optionally, monitoring of the signal may give an indication for absolute pressure values in real-time and/or relative readings over a defined period of time. According to some embodiments, the monitoring device may be able to detect the specific pressure waveforms of the venous pressure profile (a, x, v, y) that reflect the right atrial pressure and/or central venous pressure throughout the cardiac contraction cycle. Optionally, the monitoring device may provide additional information on the clinical status of various cardiac conditions such as, severity of mitral regurgitation as indicated by the V wave, right ventricular failure and pressures, follow up of pulmonary embolism patients, etc.

According to some embodiments, the monitoring device may detect an arterial pressure wave, monitor blood pressure, and/or integrate arterial and venous data. According to some embodiments, this may facilitate relative or absolute measurement of arterial blood pressure. According to some embodiments, this may facilitate analysis of the arterial pressure wave providing clinical insights which may be associated with and/or additive to the venous readings. According to some embodiments, monitoring venous pressure may enable monitoring of additional clinical scenarios, such as intravascular volume status, cardiac tamponade, pulmonary embolism, right heart failure, pulmonary hypertension, valvular disorders and/or diseases, etc.

According to some embodiments, the device may be provided as part of a kit. Optionally, the kit may include the monitoring device, an instructional leaflet, and/or a patient data acquisition processing system and/or access to a patient data acquisition processing system, e.g., cables, passwords, etc. and/or one or more spare parts, e.g., additional patches, etc. Optionally, the instructional leaflet may include information on the use, positioning and/or activation of the monitoring device and/or connection to the patient data acquisition processing system.

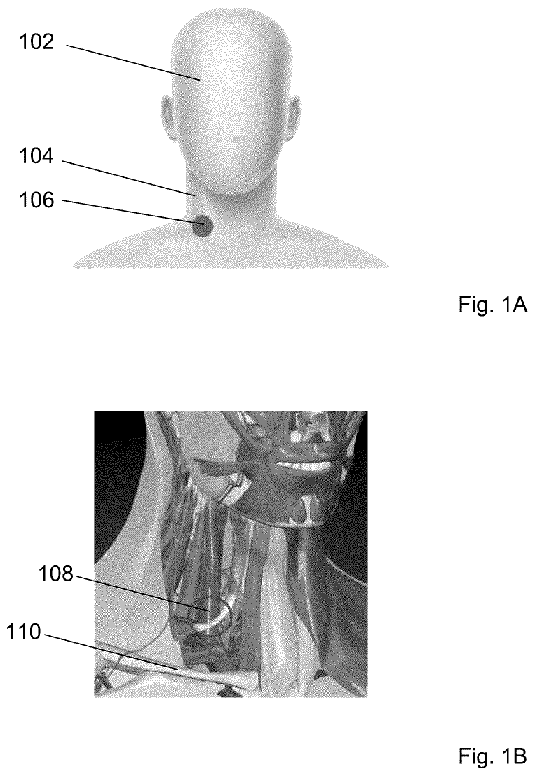

Reference is now made to FIGS. 1A and 1B, which schematically illustrate a preferred target area for pressure sensing using a central venous pressure monitoring device, in accordance with some embodiments. Target position 106, 108 for the monitoring device may be on the right internal jugular vein of the skin of a neck 104, below a head 102 and above a collar bone 110 of a user. Additionally, and/or alternatively, in case of clinical need, the left Jugular vein and/or other vascular sites may be monitored.

According to some embodiments, the monitoring device may measure skin sub-surface strain, skin fluctuations that result from vascular changes in diameter due to variation of internal pressure and/or skin stretching (pulse/beats) and/or counter response of an adhesive patch and the height of detected variations in the patient's skin in the neck at which the signal is detected. According to some embodiments, the microneedles may pierce through the stratum corneum to better detect sub-surface skin deformation due to jugular vein pressure variation.

According to some embodiments, the device may be used for monitoring pathologies characterized by changes in central venous pressure including heart failure, pulmonary embolism, pericardial tamponade, hemodynamic shock, right heart failure, valvular disorders and/or diseases.

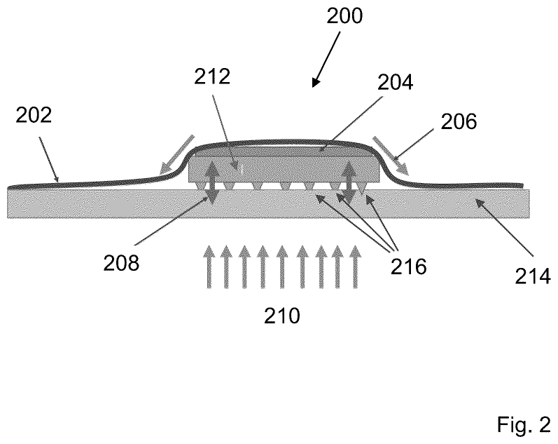

Reference is now made to FIG. 2, which schematically illustrates a side view of a central venous pressure monitoring device 200, in accordance with some embodiments. As shown herein, monitoring device 200 includes a microneedle structure 212, which includes, on one surface thereof, multiple microneedles 216, attached thereto or formed therewith. Microneedles 216 are configured to penetrate the subject's skin target area 214 e.g., for better detection of skin sub-surface deformation due to the pressure variation. Monitoring device 200 also includes a force sensor 204, associated with a second surface (opposing the first surface) of the microneedle structure. The force sensor may be in contact with the microneedles and/or may be placed within the microneedles themselves and/or embedded into the base of the microneedles.

According to some embodiments, monitoring device 200 also includes patch 202 which is configured to adhere monitoring device 200 skin target area 214. Monitoring device 200 is thus configured to monitor fluctuations 208 and/or skin stretching (beats) 210 and/or counter response 206 of the patch and/or the height in the skin at the neck at which the signal is detected.

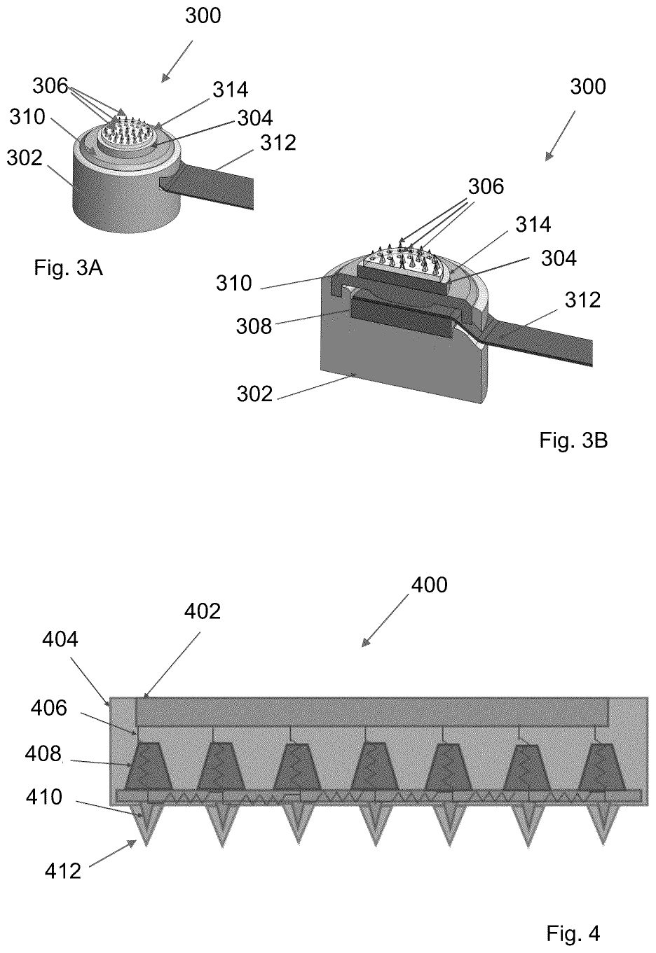

Reference is now made to FIGS. 3A-3B, which schematically illustrate a perspective view and cut away view, respectively, of a central venous pressure monitoring device 300, in accordance with some embodiments. Monitoring device 300 includes a microneedle structure 304 for a plurality of microneedles 306. The microneedle structure 304 includes a first surface to which the plurality of microneedles are attached to or integrally formed with, and a second surface optionally attached to an adhesive layer 314. Optionally, the adhesive layer 314 may include a polyurethane adhesive. The adhesive layer may be a dual adhesive, wherein a first surface may adhere to the skin of a subject and a second surface, which may adhere to a first surface of a flexible pad 310. Optionally, the flexible pad 310 may include silicone material. A second surface of a flexible pad 310 is associated with a force sensor 312 and/or a rigid substrate 308. Optionally all or part of the components described herein may be incorporated into casing 302. Optionally, the rigid substrate 308 and/or the casing 302 may be composed, at least in part, of a 3D printed material. Optionally, the microneedles 306 may be composed, at least in part, of PCL, PLGA, and/or a combination thereof. The force sensor 312 is connected by a cable and/or wirelessly to a patient data acquisition processing system.

According to some embodiments, the sensor may be a force sensor, a pressure sensor or a strain gauge, or any combination thereof. Optionally, the sensor may be an off-the shelf or specifically designed for the monitor. According to some embodiments, the sensor may be integrated inside the microneedles backing layer e.g., to enhance sensitivity. According to some embodiments, the sensor may be attached from the outside of the device.

According to some embodiments, the sensor may be manufactured from conductive stretch sensitive materials, such as graphene (and/or other carbon-based materials), silver, silicone, and more, and any combination thereof. According to some embodiments, the sensor may be manufactured using spray printing, screen printing, photolithography, other methods, or any combination thereof. Additionally, and/or alternatively, the sensor may include optic fibers and/or conductive polymers.

According to some embodiments, the sensor components may be integrated into the microneedles structure that pierces the skin, facilitating monitoring of strain changes at higher resolutions. According to some embodiments, multi-layer and/or multi-component sensor may provide high spatial resolution readings, especially when integrated with the microneedles.

Reference is now made to FIG. 4 is a schematic illustration of a central venous pressure monitoring device 400, in accordance with some embodiments. Monitoring device 400 includes sensors 410 (for example, but not limited to, a polymeric sensor) integrated with a plurality of microneedles 412 to provide a combined microneedle-mechanical sensor. The combined microneedles 404 include sensor membrane 402, resistors/capacitors 406, one or more strain elements 408, and one or more nanoparticles embedded within the microneedles, these elements may change their electrical properties (e.g., resistance, capacitance, etc.) under deformation due to pressure variation. The detection of this deformation along with appropriate calibration may facilitate the precise determination of the pressure profile variation.

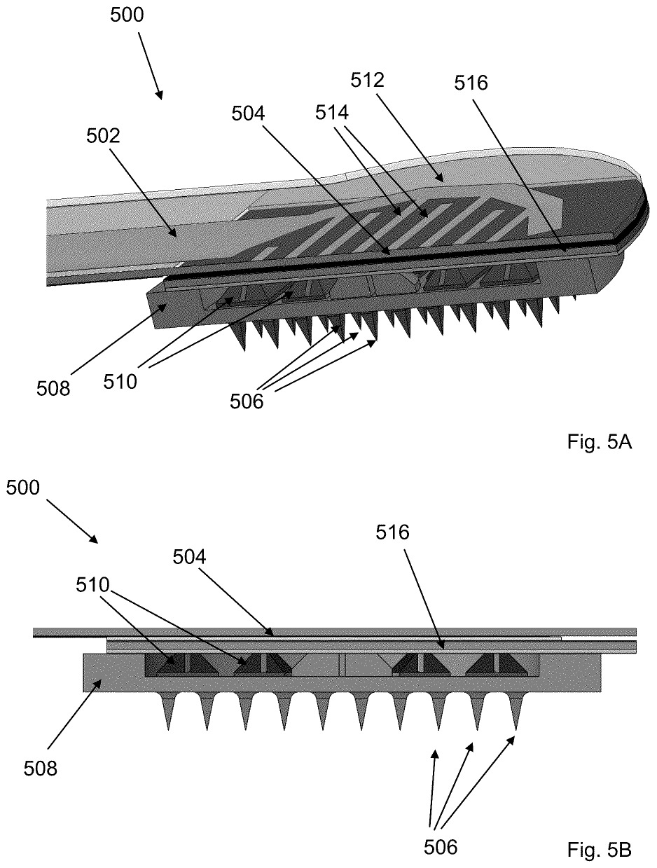

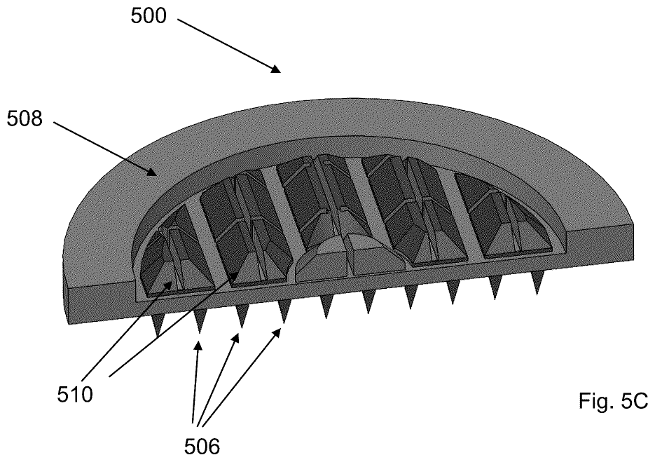

FIGS. 5A and 5B are schematic illustrations of cross sections of a central venous pressure monitoring device 500, and FIG. 5C is a schematic illustration of an isometric partial view of a central venous pressure monitoring device 500, in accordance with some embodiments. Monitoring device 500 includes a plurality of microneedles 506 supported on a microneedle structure 508, which includes one or more sensors 510, a casing 512, and optionally, a cable 502. Optionally, cable 502 may be connected to one or more sensors 510 through one or more resistors/capacitors 514. Optionally, the microneedle structure 508 is associated with a flexible pad 516 and/or a rigid substrate 504.

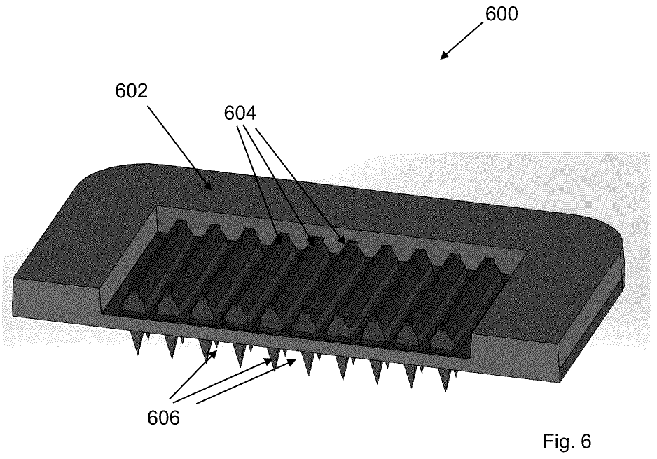

Reference is now made to FIG. 6, which is a schematic illustration of an isometric partial view of a central venous pressure monitoring device 600, in accordance with some embodiments. Monitoring device 600 includes a baking layer 602, comprised of protrusions 604 that isolate the mechanical effect associated with strain, compression or movement of one or more microneedles 606.

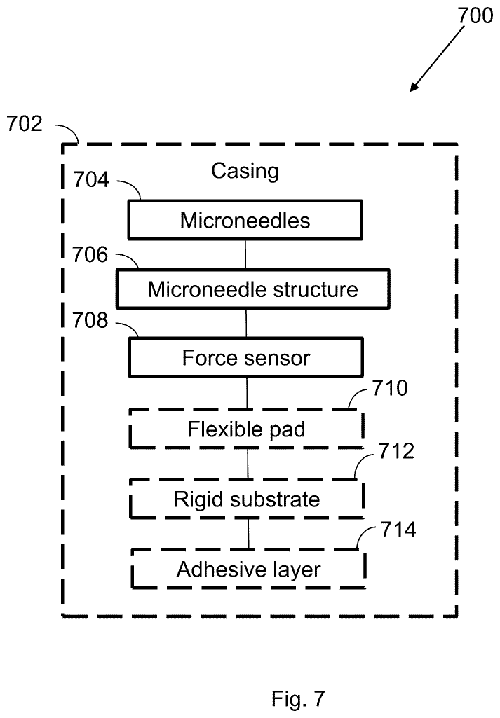

Reference is now made to FIG. 7, which is an exemplary block diagram of a central venous pressure monitoring device, in accordance with some embodiments. According to some embodiments, the monitoring device 700 may optionally include a casing 702, a microneedle structure 706, with microneedles 704 on a first surface and a force sensor 708 associate with a second surface. The device may optionally include a rigid substrate 712, a flexible pad 710, an adhesive layer 714. Optionally, the adhesive layer may be adjacent to the first and/or second surface of the microneedle structure.

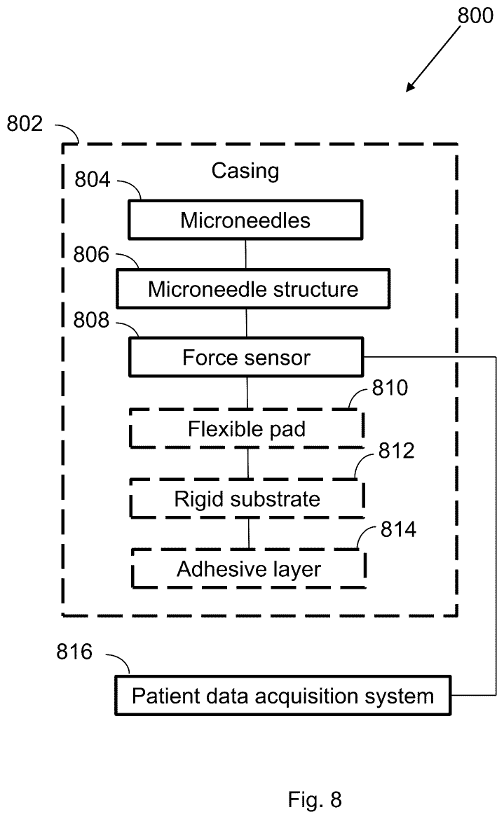

Reference is now made to FIG. 8, which is an exemplary block diagram of a central venous pressure monitoring system, in accordance with some embodiments. According to some embodiments, the system 800 may include a monitoring device and a patient data acquisition processing system 816. The monitoring device 802 may optionally include a casing. The monitoring device 802 may include a microneedle structure 806, with microneedles 804 on a first surface and a force sensor 808 associate with a second surface. The device may optionally include a rigid substrate 812, a flexible pad 810, and an adhesive layer 814. Optionally, the adhesive layer may be adjacent to the first and/or second surface of the microneedle structure. The force sensors 808 may transfer data to a patient data acquisition processing system 816, directly, indirectly, or both. Optionally, the patient data acquisition processing system 816 may receive data via a cable, or wirelessly (e.g., short-range, like Bluetooth®, or wi-fi, LAN, etc.).

### EXAMPLES

### 1. Proof-of-Concept Model

An artificial neck vein model was designed and manufactured from materials that simulate, as close as possible, the mechanical properties of the human anatomy and venous flow. The artificial model included casted silicones, pulsatile pumps, specialized tubes, and gels.

According to some embodiments, the artificial neck model may be an artificial neck arteria-vein model that simulates, as close as possible, the mechanical properties of the human anatomy and arteria and venous flows.

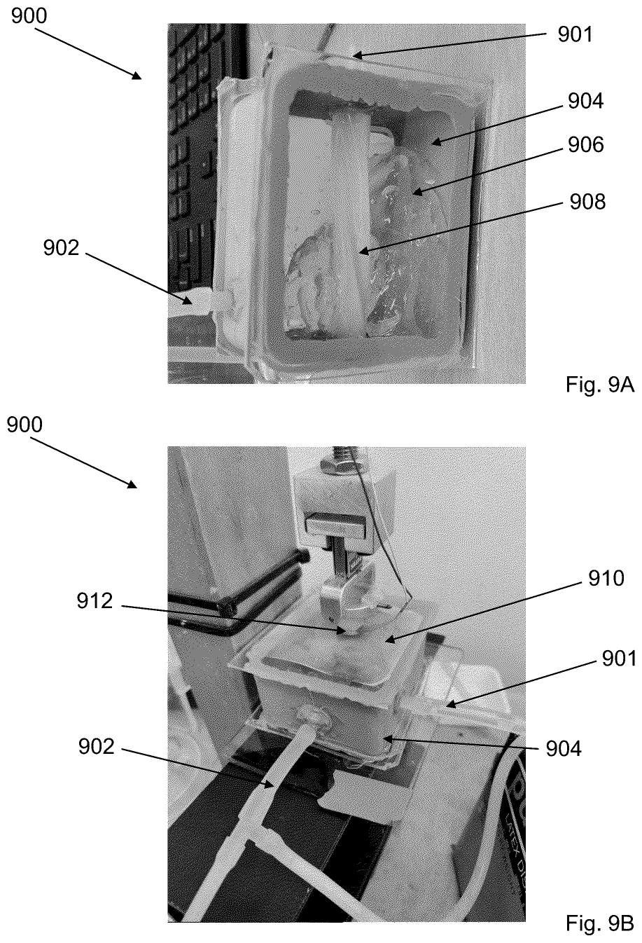

Reference is now made to FIGS. 9A and 9B, which are exemplary photographs of an artificial neck vein model 900, in accordance with some embodiments. According to some embodiments, the artificial neck vein model 900 includes a casing 904, one or more tubes 908, and a gel 906, wherein the tubes are connected to an inlet 902 and an outlet 901. Casing 904 may be covered by one or more layers 910 (e.g., soft elastomers, etc.) to simulate the skin, through which the monitoring device 912 may measure a pressure profile of the system.

The pressure monitor, the microneedles and pressure sensor (e.g., Force Sensitive Resistor) were positioned facing the artificial neck vein model “skin”, mounted on a customized test-rig that allowed application of a constant vertical force against the artificial neck vein model 900, to neutralize and standardize the effects of the skin attachment component.

Another artificial neck model was produced for simulation purpose (not shown), which includes the carotid artery and jugular vein with relevant pulses.

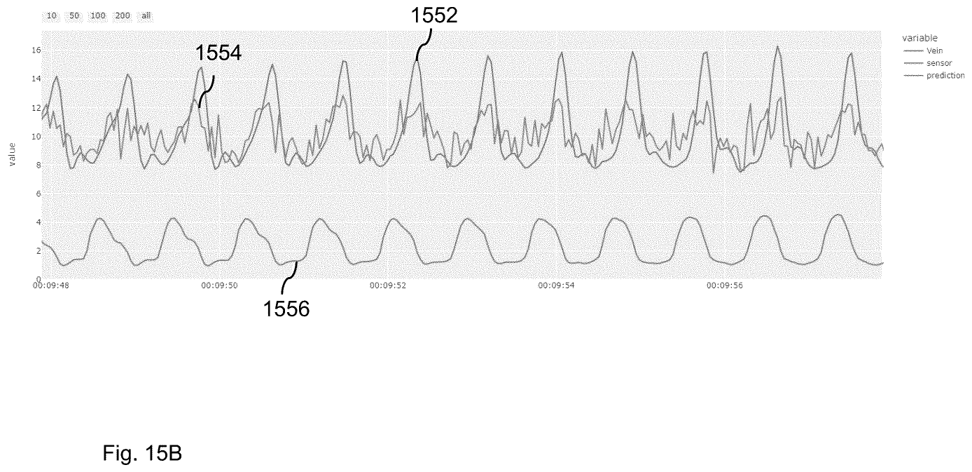

According to some embodiments, an algorithm and the device design were adjusted and trained on a such simulators (particularly the simulator that incorporates an arterial and venous pulsations) since the jugular area pulsations are mainly related to the arterial pulsations. Embodiments of this disclosure thus further relate to the extraction of the minute venous pulsation from the combined signal (see results in FIGS. 15A and 15B).

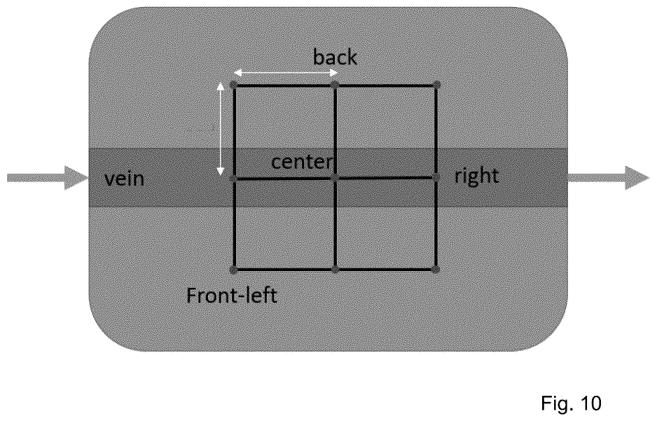

Reference is now made to FIG. 10, which schematically illustrates an upper view of a vein model with the different test locations of the monitor, in accordance with some embodiments. The target position of the monitoring device was the jugular vein. Even if the monitoring device was positioned slightly off center (e.g., during self-application, application by an inexperienced carer, etc.), if it was located within a distance of the jugular vein, a pressure profile may be measured. Optionally, the off-center device may be positioned at a distance with a radius of about 1 cm of the vein.

### Experimental Setup

The device was mounted on a vertical moving stage of the test-rig, which was driven down at a velocity of 0.8 mm/s, this caused the normal force to increase progressively until the value of 20 N was obtained. Then, the system was kept under this loaded state for a dwell time of 30 s, afterwards the moving stage was withdrawn upward at a velocity of 0.5 mm/s until a preload force of 0.5 N was achieved and the system was maintained under this loaded state during the rest of the test.

The neck model was attached to an external pulsatile pump to simulate the venous flow (pulse amplitude ranging of 2-6 mmHg), while the pressure monitor was attached to the skin model and recorded the data (pressure profile). To evaluate the sensitivity and accuracy of the pressure monitor prototype, the test was performed at four different positions with respect to the artificial vein, i.e., center, back, right, front-left (e.g., FIG. 10).

### Results

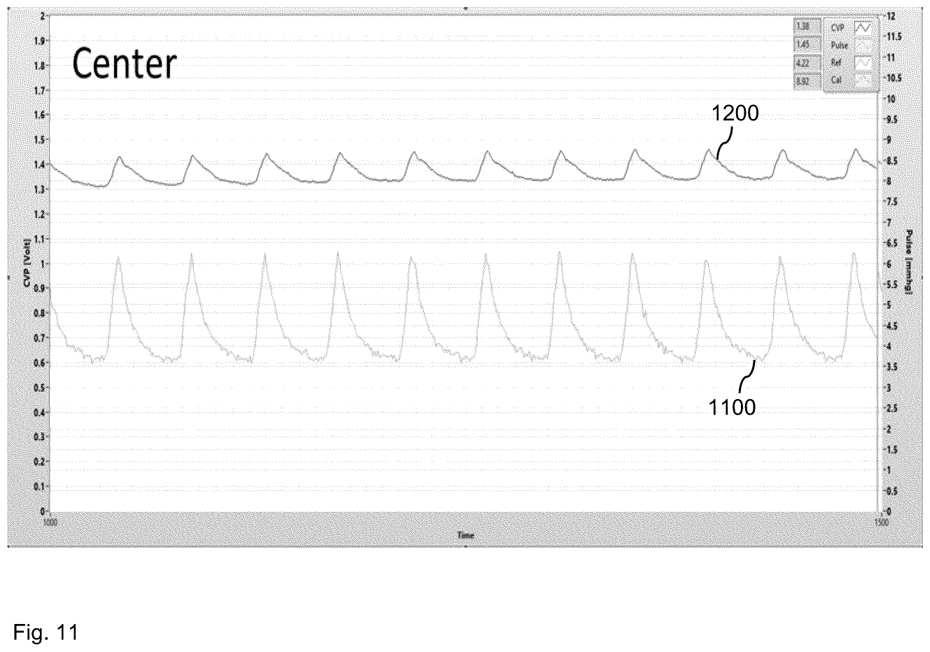

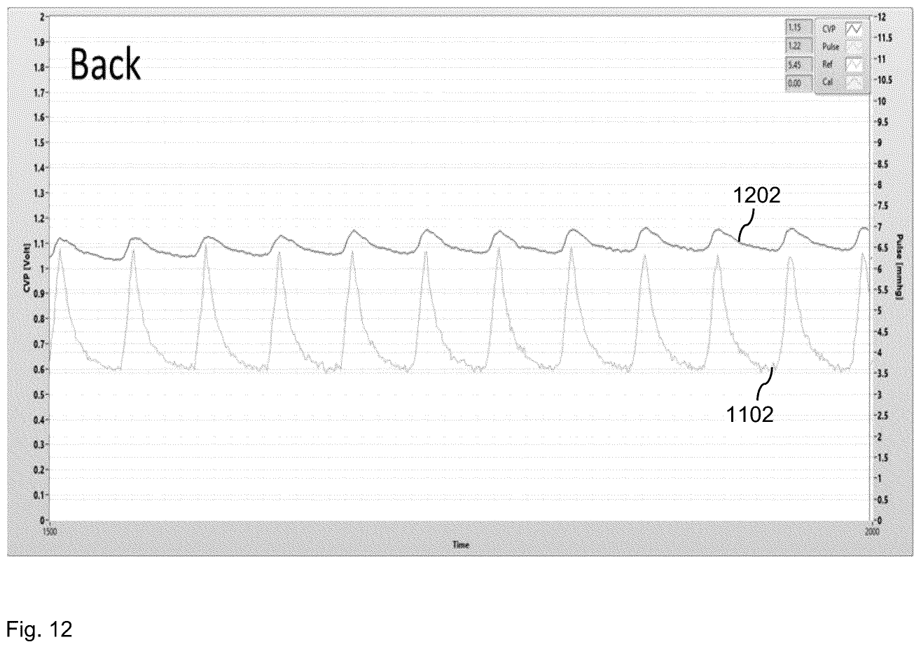

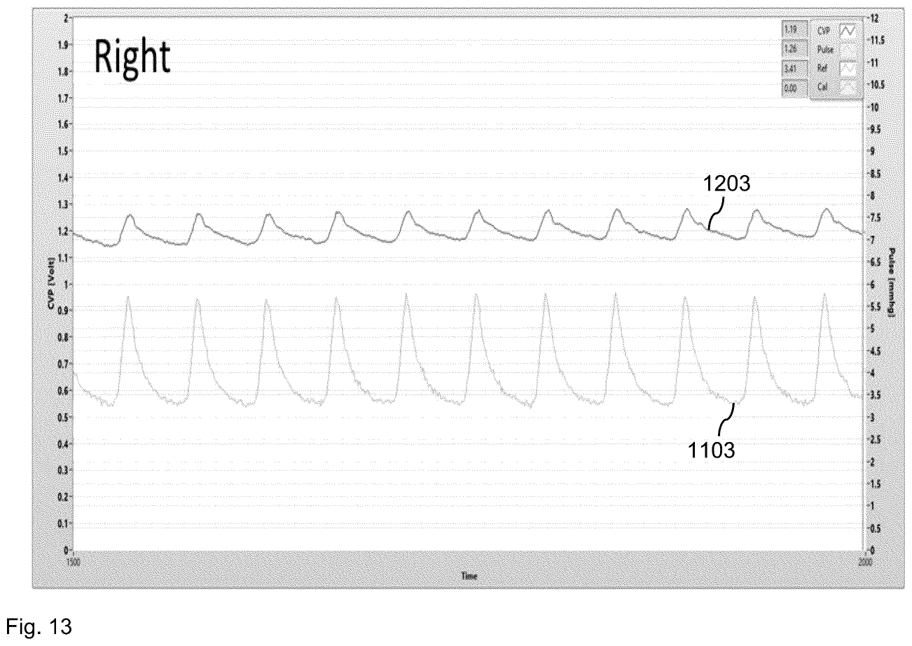

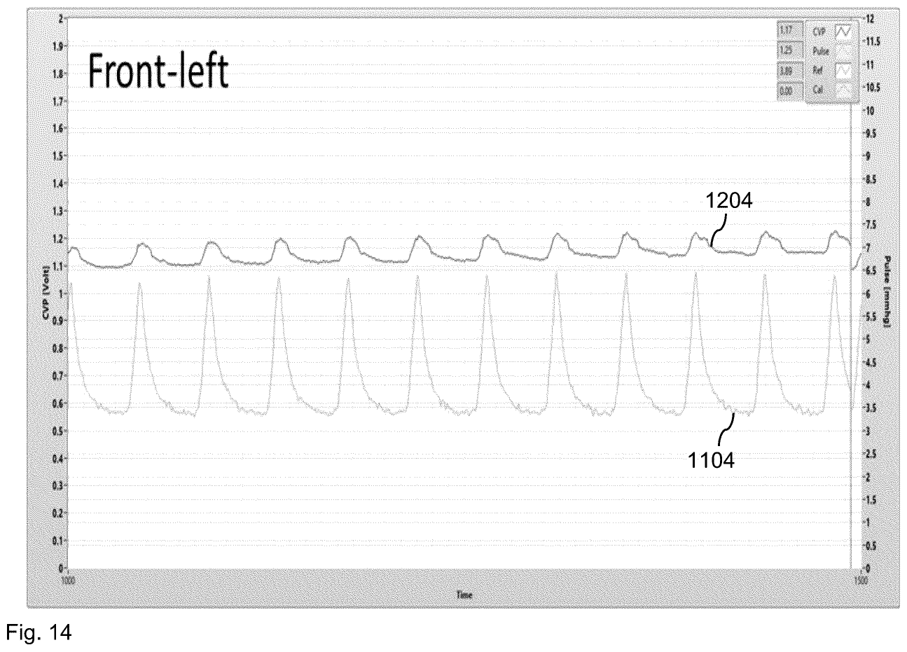

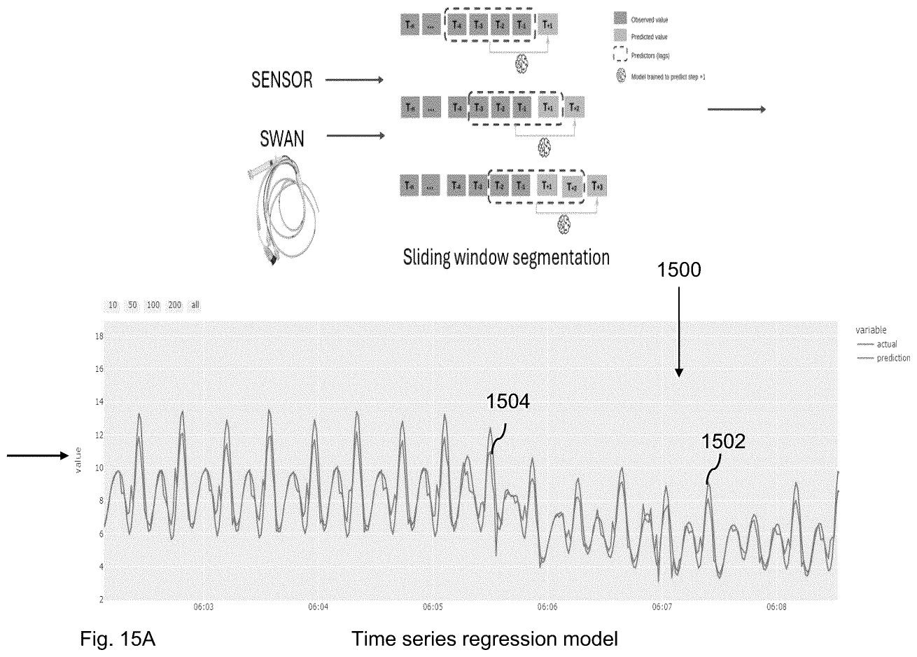

FIGS. 11, 12, 13 and 14 display pressure profiles of a central, back, right, and front-left test locations of the model flow system using a central venous pressure monitoring device, respectively. In FIG. 11, the lower line 1100 represents the actual pressure in the model flow system delivered by the pump in mmHg (right axis), while the upper line 1200 represents the force recorded by the novel prototype of pressure monitor in voltage (left axis). In FIG. 12, the lower line 1102 represents the actual pressure in the model flow system delivered by the pump in mmHg (right axis), while the upper line 1202 represents the force recorded by the novel prototype of pressure monitor in voltage (left axis). In FIG. 13, the lower line 1103 represents the actual pressure in the model flow system delivered by the pump in mmHg (right axis), while the upper line 1203 represents the force recorded by the novel prototype of pressure monitor in voltage (left axis). In FIG. 14, the lower line 1104 represents the actual pressure in the model flow system delivered by the pump in mmHg (right axis), while the upper line 1204 represents the force recorded by the novel prototype of pressure monitor in voltage (left axis). The device clearly detected the pulsatile waves pressure variation of the simulation model and successfully recorded the pressure signals in all positions with good sensitivity.

To optimize the patient data acquisition processing system (or the processor) of the above-mentioned pressure monitoring device, an algorithm for data analysis has been developed. The data for developing the algorithm for data analysis was created in two stages. The first stage was conducted on a simulator that mimics a neck with blood vessels designed to represent the carotid artery and jugular vein, incorporating the mechanical properties of the relevant blood vessels and tissues (similarly to the above-mentioned neck model). A simulation of blood flow through the simulator was performed while measuring the pressure inside the vein, alongside simultaneous pressure measurements with the herein disclosed central venous pressure monitoring device. The integration of this data was used for the first stage of algorithm (e.g., Artificial Intelligence (AI) based algorithm) development. Subsequently, data from an approved clinical trial with a similar design was collected in humans undergoing right heart catheterization as part of their clinical evaluation. Data were collected simultaneously from the invasive catheter (Swan-Ganz) and from the herein disclosed central venous pressure monitoring device (sensor).

Reference is now made to FIG. 15A, which schematically illustrates the development of a time series regression model for predicting central venous pressure, in accordance with some embodiments. The input from the herein disclosed central venous pressure monitoring device (SENSOR) and from the Swan-Ganz (SWAN) catheter was taken for each timestamp. There were 60 samples per second. The Swan-Ganz data was regarded as the dependent variable and the herein disclosed central venous pressure monitoring device's data was regarded as the independent variable. Thereafter, the data was entered to a linear regression machine learning model with sliding window segmentation. The final output was a time series regression model 1500. Graph line 1502 represents the actual pressure, while line 1504 represents the model-based predicted values. A good correlation between line 1502 and 1504 is observed.

Reference is now made to FIG. 15B, which shows a graph of true venous pressure readings 1552 (using Swan) compared to the algorithm's prediction values 1554 as derived from the sensor reading 1556 using a simulator that integrates a model of the carotid arterial and jugular venous vessels. A good correlation between the true venous pressure reading 1552 and the algorithm's prediction values 1554 is observed.

Advantageously, as can be seen from the results disclosed hereinabove, pressure values obtained by the herein disclosed device/system for monitoring the subject's central vein pressure utilizing the algorithm are indicative of the actual central venous pressure. According to some embodiments, the trained algorithm is configured to produce pressure values which are devoid of arterial or other undesired effects on the measured values (signals).

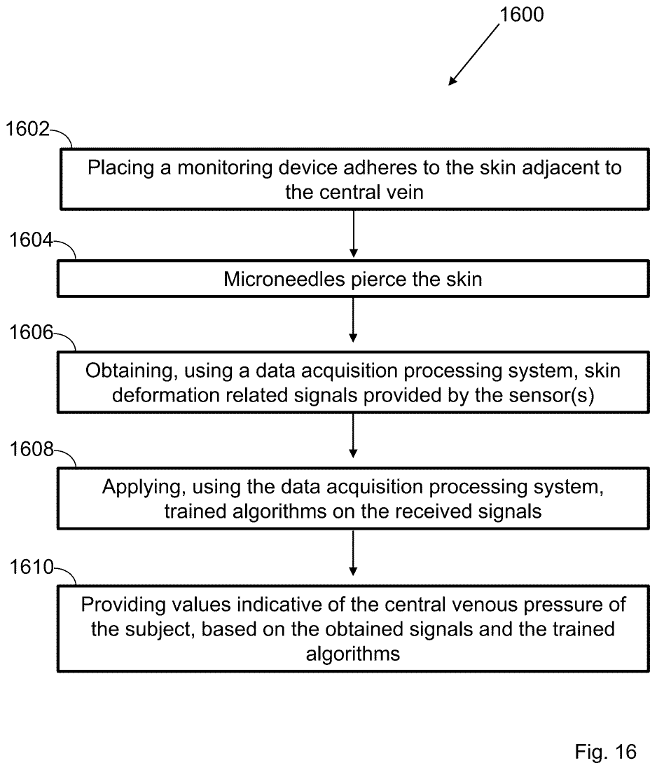

Reference is now made to FIG. 16, which is an illustrative flowchart of a method for monitoring central venous pressure using a monitoring device, according to some embodiments. According to some embodiments, method 1600 may include placing a monitoring device to the skin adjacent to the central vein (e.g., jugular vein) (1602). Piercing through the stratum corneum of the skin using the device's microneedles structure (1604). Obtaining, using a data acquisition processing system, skin deformation related signals provided by the sensor(s) (1606). Applying, using the data acquisition processing system, trained algorithms on the received signals (1608), and providing values indicative of the central venous pressure of the subject, based on the obtained signals and the trained algorithms (1610).

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The terms “comprises”, “comprising”, “includes”, “Including”, “having” and their conjugates mean “including but not limited to”.

The term “consisting of”means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 80% and 120% of the given value.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purposes and should not be regarded as limiting. Citation or identification of any reference in this application shall not be construed as an admission that such reference is available as prior art to the disclosure. Section headings are used herein to ease understanding of the specification and should not be construed as necessarily limiting.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

The following examples are presented to provide a more complete understanding of the invention. The specific techniques, conditions, materials, proportions and reported data set forth to illustrate the principles of the invention are exemplary and should not be construed as limiting the scope of the invention.

1. A device for essentially non-invasively monitoring a subject's central venous pressure comprising:
a microneedle structure comprising a first surface, a second surface opposing said first surface, and a plurality of microneedles attached to or being an integral part of said first surface, wherein the plurality of microneedles is configured to penetrate a skin surface at a target area above and/or in vicinity to the subject's central vein; and
at least one sensor associated with or incorporated within the microneedle structure,
wherein the at least one sensor is configured to provide signals indicative of the central venous pressure.

2. The device according to claim 1, further comprising a flexible pad configured to conform the microneedle structure to contours of the subject's target area.

3. The device according to claim 2, wherein the flexible pad is positioned in proximity to the second surface of the microneedle structure.
The device according to claim 1, further comprising an adhesive layer.

4. The device according to claim 4, wherein the adhesive layer is a double adhesive layer located between the second surface of the microneedle structure and a first surface of the flexible pad.

5. The device according to claim 4, wherein the adhesive layer extends over and beyond a second surface of the flexible pad and is configured to attach the device to the subject's skin.
The device according to claim 1, further comprising a rigid substrate positioned at the base of the device, wherein the rigid substrate is configured to generate contralateral pressure to the venous pressure and increase the sensor sensitivity.

6. (canceled)
The device according to claim 1, wherein the device is associated with a patient data acquisition processing system configured to receive data from the at least one sensor.

7. (canceled)

8. (canceled)
The device according to claim 1, wherein the at least one sensor comprises a force sensor, a pressure sensor or a strain gauge, wherein the at least one sensor is configured to detect skin sub-surface strain, skin fluctuations that result from vascular changes in diameter due to variation of internal pressure, skin stretching, or a combination thereof.

9. (canceled)

10. The device according to any one of claim 1, further comprising a casing configured to house the microneedle structure and the at least one sensor.

11. The device according to claim 14, wherein the casing further comprises a flexible pad, a rigid substrate, a connection to a patient data acquisition processing system or any combination thereof.
The device according to claim 1, wherein the density of the microneedles on the first surface of the microneedle structure is between about 5 to about 150 microneedles/cm².

12. (canceled)
The device according to claim 1, wherein the at least one sensor is integrated within the microneedles to provide a combined microneedle-mechanical sensor.

13. (canceled)

14. (canceled)

15. (canceled)

16. A system for essentially non-invasively monitoring a subject's central venous pressure, the system comprising:
a microneedle structure comprising a first surface and a second surface opposing said first surface, wherein the microneedle structure further comprises a plurality of microneedles attached to or integrated within the first surface thereof, and wherein the plurality of microneedles is configured to penetrate a skin surface at an area above and/or in vicinity to the subject's central vein;
at least one sensor associated with or incorporated within the microneedles and/or the microneedle structure, wherein the at least one sensor is configured to provide pressure indicative signals; and
a patient data acquisition processing system configured to receive data from the at least one sensor and to apply trained algorithms on the received pressure signals, and to provide values indicative of the central venous pressure of the subject.

17. (canceled)

18. (canceled)

19. (canceled)

20. A method for essentially non-invasively monitoring the central venous pressure of a subject, the method comprising:
placing a central venous pressure monitoring device to the subject's skin surface at an area above and/or in vicinity to the subject's central vein, wherein the device comprises:
a microneedle structure comprising a first surface, a second surface opposing said first surface, and a plurality of microneedles attached to or being an integral part of the first surface thereof; and
at least one sensor in association with or incorporated within the microneedles and/or the microneedle structure;
piercing the skin surface with the microneedles;
measuring skin deformation with the at least one sensor;
obtaining, using a data acquisition processing system, skin deformation related signals;
applying, using the data acquisition processing system, trained algorithms on the received signals; and
providing values indicative of the central venous pressure of the subject, based on the obtained signals and the trained algorithms.

21. The method of claim 26, further comprising transmitting the signals to a patient data acquisition processing system.

22. (canceled)
The method of claim 26, further comprising providing a central venous pressure profile of the subject.

23. The method of claim 29, further comprising providing the pressure profile to a medical professional for real-time monitoring and/or periodic review.

24. (canceled)

25. (canceled)

26. (canceled)

27. (canceled)
The method of claim 26, wherein the monitoring comprises monitoring any one of fluctuations in venous pressure, and specific pressure waveforms arising from the central vein.

28. (canceled)
The method of claim 26, further comprising predicting central venous pressure based on measured sub-surface skin deformation and a trained algorithm model.

29. (canceled)
